(19) United States
(12) Patent Application Publication
(10) Pub. No.: US 2025/0305015 A1
(43) Pub. Date: Oct. 2, 2025

(54) MULTIFUNCTIONAL NANOPARTICLES IN PLANTS

(71) Applicant: Colin Theodore Shepherd, Brooklyn, WI (US)

(72) Inventor: Colin Theodore Shepherd, Brooklyn, WI (US)

(73) Assignee: NANOVITICS INC, VERONA, WI (US)

(21) Appl. No.: 18/623,580

(22) Filed: Apr. 1, 2024

(51) Int. Cl. C12P 17/18; C12N 9/02; C12N 15/82

(52) U.S. Cl. CPC C12P 17/182; C12N 9/0071; C12N 15/8205; C12N 15/825; C12Y 114/18001

(57) ABSTRACT

This invention relates to the production of plant-made melanin nanoparticles. This comprises utilizing the agrobacterium infiltration of a plant or plant cell to initiate a plant cell response by way of tyrosinase enzymatic or similar biochemical reactions to form biosynthetic plant-made melanin nanoparticles. The plant-made melanin nanoparticles can be utilized for biomedical, healthcare, therapeutic, environmental, or industrial use among many other uses.

### CROSS-REFERENCE TO RELATED APPLICATIONS

This application claims priority under 35 U.S.C. § 119 to U.S. Provisional Patent Application No. 63/497,968, filed Apr. 24, 2023, which is incorporated herein by reference.

### BACKGROUND OF THE INVENTION

Nanoparticles are increasingly being utilized in a variety of industries including biomedicine, environmental technologies, agriculture, manufacturing, biotechnology, healthcare, and many others. Melanin is a natural nanoparticle substance produced by microorganisms, plants, and animals that exhibits a multifunctional array of unique physical and chemical properties. These include ultraviolet and visible light absorption, photothermal conversion, ultraviolet protection, radical scavenging, immunomodulatory activities, and natural biocompatibility. Due to these and other properties, and its capability to be produced using a biosynthetic process, Melanin nanoparticles and nanomaterials have potential wide uses in numerous platform-based applications across many important industries.

Many limitations exist for current nanoparticles. Metal nanoparticles such as gold nanoparticles are difficult to produce and cannot be manufactured using a biosynthetic process. Increasing the scale of production of nanoparticles is difficult and limited due to the synthetic nature of manufacture, the cost of raw materials, and batch to batch inconsistencies. Many of the chemicals used in the manufacture of synthetic metal nanoparticles are toxic, difficult to produce and dispose of, and are hazardous to personnel involved in the manufacturing process. Many nanoparticles themselves are not environmentally friendly to use or dispose of. In addition, many current nanoparticles are not biocompatible, and this severely limits their use in healthcare, biomedical, and environmental applications.

Melanin nanoparticles and nanomaterials also have current limitations in production and manufacture. In most cases, natural melanin is extracted from organisms using difficult processes, hazardous chemicals, and often by harvesting organisms from the natural world using environmentally damaging and unethical methods. Synthetic melanin can be manufactured but the process is not easily scalable. Furthermore, the biocompatibility of synthetic melanin is not as robust as biosynthetic melanin and hinders its potential uses. Biosynthetic melanin nanoparticles, meaning nanoparticles made from biological systems using biotechnology, have many advantages over other nanoparticles and are undergoing intensive research, but supplies of biosynthetic melanin are limited in scale, quality, and use. Vast improvements need to be made in production, manufacture, and quality before these unique nanoparticles become widely available. Plants offer unique advantages in economy, efficiency of production, and quality for nanoparticle production that needs to be explored.

There is a clear unmet need for the production and manufacture of high quality, scalable, biosynthetic, sustainable, and environmentally friendly melanin nanoparticles for downstream use in a variety of industries including biomedicine.

### SUMMARY OF THE INVENTION

Therefore, it is an objective of the current invention to produce and manufacture multifunctional melanin nanoparticles in plants, plant tissues and seeds and roots, and plant extracts, termed plant-made melanin nanoparticles. These plant-made melanin nanoparticles are useful for biomedical, industrial, agricultural, and many other uses. A further objective is to improve the creation and production of plant-made multifunctional melanin nanoparticles for multiple downstream uses.

A first aspect of the invention is the genetic expression of a tyrosinase enzyme in plants, plant tissues and seeds, plant cells, plant roots, and plant extracts.

A further aspect of the present invention is the ultimate creation of plant-made melanin nanoparticles by way endogenous proteins or biochemicals or recombinant plant-made tyrosinase or similar proteins and endogenous or supplied cofactors in a plant extract.

A further aspect of the present invention is the precipitation of plant-made melanin nanoparticles from plant extract either by natural or chemical process with plant-made melanin nanoparticles purified into deionized, distilled water byway of centrifugation or filtration.

A further aspect of the present invention is the utilization of plant-made melanin nanoparticles for downstream uses including, but not limited to, biomedical imaging, healthcare, photothermal therapy, diagnostics, industrial uses, environmental uses, and agricultural uses.

A further aspect of the present invention is the increased functionalization of the plant-made melanin nanoparticles by attaching, incorporating, or encapsulating a chemical, a biochemical, a protein, or another material that creates a multifunctional plant-made melanin nanoparticle.

The preceding is a simplified summary to provide an initial understanding of the aspects, embodiments and configurations disclosed herein. This summary is neither an extensive nor exhaustive overview of the aspects, embodiments, or configurations. It is intended neither to identify key or critical elements, nor to delineate the scope of the aspects, embodiments, or configurations but to present selected concepts in a simplified form as an introduction to the more detailed description presented below. As will be appreciated, other aspects, embodiments, and configurations are possible utilizing, alone or in combination, one or more of the features set forth above or described in detail below.

### BRIEF DESCRIPTION OF DRAWINGS

The accompanying drawings are incorporated into and form a part of the specification to illustrate examples of how the aspects, embodiments, or configurations can be made and used and are not to be construed as limiting the aspects, embodiments, or configurations to only the illustrated and described examples. Further features and advantages will become apparent from the following, more detailed description of the various aspects, embodiments, or configurations.

FIG. 1 illustrates one embodiment and is a diagram of the method and process to produce plant-made melanin nanoparticles.

FIG. 2 illustrates one embodiment and is a line chart showing the results of UV-VIS spectra of a sample of plant-made melanin nanoparticles compared to a plant extract control.

### DETAILED DESCRIPTION OF THE INVENTION

The following detailed description illustrates the invention by way of example and not by way of limitation. This description will clearly enable one skilled in the art to make and use the invention.

The present invention relates to the production, manufacture, and utilization of plant-made melanin nanoparticles. Potential uses for embodiments of the present invention include, but are not limited to, biomedicine, agriculture, radical scavenging, environmental remediation, photoacoustic imaging, medical treatments and therapies, and photodynamic therapies.

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

In one aspect, the present invention is directed a plant-made melanin nanoparticle produced in a plant or plant extract. As will be understood by those skilled in the art, “melanin nanoparticle” has come to refer to a broad range of black heterogeneous polymers, and as set forth above, can be broadly categorized based on their structure and monomer units such as eumelanin, among others, and generally exist in the ultra-small realm of 5-200 nm in diameter.

An aspect of the present invention is an agrobacterium infiltration of plant cells with a DNA construct containing a tyrosinase gene which therefore begins the production of tyrosinase (E.C. 1.14.18.1) or similar enzyme in plant tissues and plant cells. Recombinant plant-made tyrosinase is then able to catalyze certain endogenous or supplied biochemicals into biosynthetic plant-made melanin nanoparticles of a certain desired size and form. In one example, the plant-made melanin nanoparticles precipitate naturally from a tobacco plant extract solution forming a dark black concentration from a clear plant extract over a short period of time (30 minutes). This plant-made melanin nanoparticle concentration in plant extract can then be centrifuged or filtered, and purified with deionized, distilled water. Once purified, the plant-made melanin nanoparticles can be prepared for later use in a variety of industries and uses.

As further shown and described herein, the present invention provides an efficient and green synthesis solution to manufacture plant-made melanin nanoparticles from plants, plant tissues, plant cells, and plant cell extracts. The present invention also provides an improved yield of melanin nanoparticles and a safer method to precipitate and purify plant-made melanin nanoparticles without the usage of toxic chemicals.

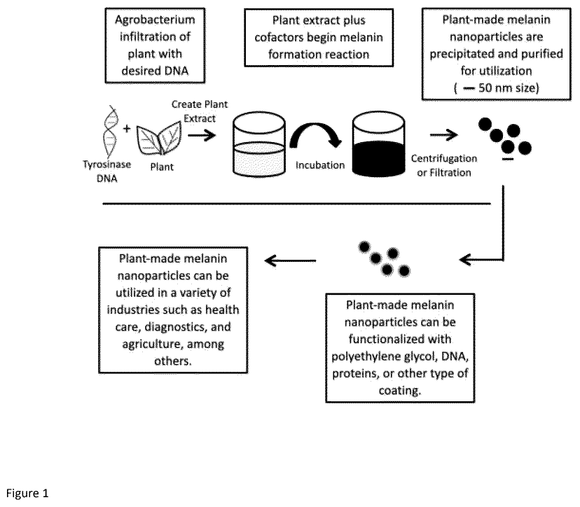

Referring to FIG. 1, a diagram illustrating the steps to produce plant-made melanin nanoparticles is provided in accordance with one or more embodiments. In one embodiment a tobacco leaf transient expression method produced biosynthetic melanin nanoparticles in plant cells and plant extracts. The method begins by agrobacterium-mediated infiltration of plant tissues resulting in endogenous release of proteins and biochemicals in the plant that, along with transient transformation of a tyrosinase-like gene, will result in the formation of plant-made melanin nanoparticles. The plant or plant tissues are incubated 3-7 days under illumination cycles for agrobacterium to infect the plant.

In some embodiments of present invention, agrobacterium infiltration of plant leaf cells instigated a plant cellular response that increased the conversion of tyrosine to intermediary biochemicals that resulted in plant-made melanin nanoparticle formation by an obvious change from clear to black color.

In some embodiments of present invention, a tyrosinase or similar genetic sequence is inserted into plants by stable genetic transformation or transient expression byway of agrobacterium infiltration or other method to insert genetic sequences into plant cells. In another embodiment the tyrosinase genetic sequence can create recombinant tyrosinase protein by cell free genetic sequence or traditional and selective plant breeding and crossing.

In some embodiments of present invention, after incubation with agrobacterium, a plant extract is created by mechanically blending or pulverizing the plant tissues. Through centrifugation or filtration, the solid plant tissue debris is removed from the plant extract, resulting in a clarified plant extract containing endogenous, recombinant, and supplied proteins and biochemicals.

In some embodiments of present invention, a tyrosinase or similar protein is expressed in plants, plant tissues, seeds, roots, and plant extracts for use as an enzyme. These enzymes convert precursors such as tyrosine, 3,4-dihydroxyphenylalanine (DOPA), and dopamine (DA) among others into major intermediaries than can be further polymerized and assembled into melanin nanoparticles in a biosynthetic manner. The timing of this enzymatic reaction can occur over varied times and temperatures to achieve the desired result of melanin nanoparticle form. The plants parts are mechanically blended into a solution and the plant extract containing tyrosinase or other biochemical or protein is clarified from plant parts by centrifugation or filtration. Centrifugation separates components based on density, with an extract sitting on top (supernatant), and a pellet collected at the bottom (precipitate), whereby the plant extract is the supernatant. Cofactors such as additional tyrosine or Copper Sulfate can be added to this clarified plant extract to aid in the formation of plant-made melanin nanoparticles.

In at least one embodiment of present invention, the plant extract is incubated at 37 Celsius for a period of time with vigorous shaking to allow plant-made melanin nanoparticles to form. This incubation time can be predetermined and will result in particular aspects of melanin nanoparticle formation such as concentration and particle size. In general plant-made melanin nanoparticle size can be controlled to 5-200 nm in diameter, but other sizes are possible by altering the incubation conditions. The plant-made melanin nanoparticle formation is obvious by a dark color appearing in the plant extract.

In some embodiments of present invention, the plant-made melanin nanoparticles are precipitated from the many forms of plant tissue, seeds, roots, extract, or buffer solution by altering the pH. The timing of this precipitation has important consequences for the size and aggregate of the plant-made melanin nanoparticles. In some cases, a short or long precipitation event can occur to give the desired size.

In some embodiments of the present invention, the plant-made melanin nanoparticles precipitate in a plant extract by a natural precipitation due to the biochemistry of the plant extract. Under this scenario, no altering of the pH is required, and this can be classified as a green synthesis method, requiring no toxic, harmful, or expensive additional chemicals to precipitate plant-made melanin nanoparticles from solution or suspension. The timing of the natural plant-made melanin nanoparticle natural precipitation in the plant extract has important consequences of for the size of the plant-made melanin nanoparticles, with a longer incubation resulting in a greater size (over 50 nm in diameter). The size of the plant-made melanin nanoparticle can also be altered by addition of H2O to change the plant extract concentration during incubation and precipitation events.

In some embodiments of the present invention, the plant-made melanin nanoparticles are formed in plant cells, plant apoplasts, plant tissues, plant leaves, plant roots, plant flowers and pollen, plant tissue culture, or plants as a whole.

In some embodiments of the present invention, the plant-made melanin nanoparticles are formed in a buffer of a plant cell culture.

In some embodiments of the present invention, the plant-made melanin nanoparticles are formed in a plant cell extract or in a plant cell free expression system.

In some embodiments of the present invention, the plant-made melanin nanoparticles can be purified from the plant extract by centrifugation at 5000 rpm for 10 minutes, washed and purified into deionized and distilled water and stored at room temperature, at freezing temperatures, or after drying or lyophilization.

In some embodiments of the present invention, the plant-made melanin nanoparticles can be purified and prepared for use in biomedical applications, and if necessary, can be manufactured by using good manufacturing practices (GMP).

In some embodiments of the present invention, the plant-made melanin nanoparticles can be coated with a chemical, a protectant, or a material such as polyethylene glycol (PEG). The surface of melanin nanoparticles contains highly reactive chemical groups such as hydroxyl, amine, and carboxyl groups that can be used as a platform for reactions containing different functional components. Through Michael addition reactions, Schiff base reactions, and other common chemical reactions such as crosslinker chemistry of carboxyl and amine groups, molecules of many types can be attached to the melanin nanoparticle, functionalizing the melanin nanoparticle for utilization in a variety of scenarios. PEG can be attached to the melanin nanoparticle and act as a protectant or stabilizer.

In some embodiments of the present invention, the plant-made melanin nanoparticles can be functionalized through Schiff base, Michael addition, and crosslinking chemistry, with DNA, RNA, aptamers, antibodies, proteins, chemicals, molecules, or other small molecules that functionalize and increase or decrease the activity or utilization of the melanin nanoparticle. In at least one embodiment, the functionalized melanin nanoparticle can be used in a lateral flow device to identify an analyte.

The plant species to which the present method can be adapted, includes but in not limited to Medicaga sp., Trifa-lium sp., Ulmus sp., Pyrus malus, Prunus armeniaca, Cynara acolymus, Asparagus oicinale, Hordeum sp., Galium sp., Bela vulgaris, Prunus serolina, Ligna sinensis, Nyssa sylvalica, Quercus sp., Arlocarpus allilis, Brassica sp., Andropogon scoparius, Fagopyrum sagillalum, Manihol esculenla, Apium graveolens, Agropyron deserlorum, Cornus orida, Phaseolus sp., Trilicum sp., Oenolhera caespilosa, Carya sp., Lacluca sp., Impatiens sp., Helianlhus sp., Ledum decum bens, Astragalus pallersoni, Selaria ilalica, Vaccinium mylrillus, Avena saliva, Pelroselinum crispum, Paslinaca saliva, Pisum sp., Prunus sp., Pyrus communis, Musa para disiaca, Astragaluspreussii, Raphanus salivus, Secalse cere ale, Sassa′as albidum, Alriplex conferlifolia, Sillandsia usneoides, Spinacia oleracea, Liquidambar slyraciua, Linaria Sriphylla, Liriodendron Zulipfera, licia sp., Cisrullus vulgaris, Melilolus sp., Salix sp., Rhus copallina, Nicotiana sp., Lisis sp., Dalura sp., Lycapersican sp., Solanum sp., Capsicum sp., Cucumis sp., Fragaria sp., Petunia sp., Geranium sp., Coleus sp., Slevia sp., Oryza sp., Nepela sp., Zea mays, Glycine max, and Arabidopsis thaliana.

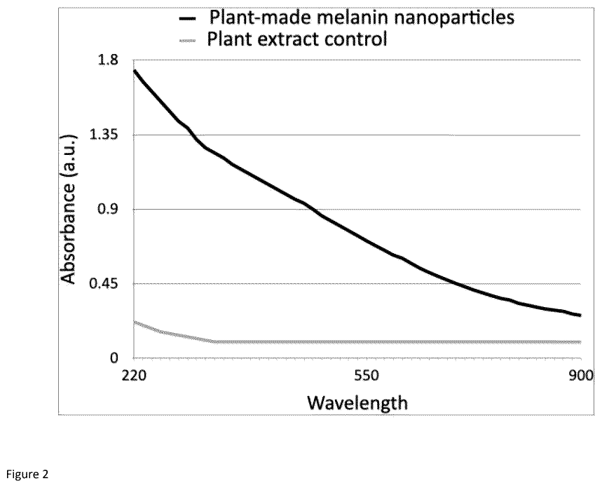

By way of example, a combination of recombinant DNA and DNA synthesis methods was used to construct each expression construct which used the 35S2 cauliflower mosaic virus constitutive promoter to drive the expression of the tyrosinase gene coupled with the nopaline synthase terminator region in the pcam2300 plant expression vector. By way of agrobacterium-mediated plant transient transformation, the DNA sequence was inserted into the plant cell and the plant leaf and allowed to incubate under lights for 5 days. After incubation, the plant leaf was mechanically blended in a ratio of 1 gram of leaf tissue to 50 ml of water, and centrifuged. The plant extract containing recombinant plant-made tyrosinase was spiked with 0.1 mM copper sulfate and 1 mM L-tyrosine and allowed to incubate at 37C with shaking. Plant-made melanin nanoparticle production can be tracked over time by formation of a dark color in the plant extract solution. The plant-made melanin nanoparticles naturally precipitated, and the precipitated melanin was centrifuged from the plant extract at 5000 rpm for 10 minutes and purified with distilled deionized water resulting in multifunctional plant-made melanin nanoparticles ready for downstream uses. A UV-vis spectrophotometer reading shown in FIG. 2 gauges the quality of the plant-made melanin nanoparticles by way of wavelength measurement of the sample (220 nm to 900 nm) and is utilized to measure nanoparticle quality against a control. The biosynthetic plant-made melanin nanoparticles were then incubated with polyethylene glycol (PEG) with shaking for 12 hours to coat the nanoparticles by the Michaels addition method.

The description above is not intended to limit the invention, as one of skill in the art would recognize from the above teachings and their accompanying examples that other techniques and methods can satisfy the claims and can be employed without departing from the scope of the claimed invention.

1. A method for the production and manufacture of plant-made melanin nanoparticles consisting of:
a agrobacterium transformation of a plant,
a tyrosinase gene inserted into a plant,
a tyrosinase enzyme expressed in a plant,
a tyrosinase enzyme that acts on precursors to form intermediates that form plant-made melanin nanoparticles,
a precursor can be cofactors or additional supplements,
a biosynthesis of plant-made melanin nanoparticles,
a precipitation and purification of plant-made melanin nanoparticles,
a use of plant-made melanin nanoparticles.

2. A method of claim 1, wherein a plant is a plant cell, plant tissue, plant root, plant seed, whole plant, plant pollen, plant extract, plant extracellular space, or cell free plant extract activated for expression of proteins by agrobacterium.

3. A method of claim 1, wherein the agrobacterium infection and infiltration of a plant cell initiates a plant response that creates the environment for plant-made melanin nanoparticles to form.

4. A method of claim 1, wherein a tyrosinase is any tyrosinase genetic sequence or similar.

5. A method of claim 1, wherein a tyrosinase enzyme is any tyrosinase enzyme (E.C. 1.14.18.1) or enzyme with activity similar to tyrosinase on intermediaries.

6. A method of claim 1, wherein plant-expressed tyrosinase enzymatically interact with supplemented intermediaries and co-factors such as L-tyrosine, copper sulfate, DOPA, DA, but not limited to these.

7. A method of claim 1, wherein a biosynthetic plant-made melanin nanoparticle is formed.

8. A method of claim 1, wherein the plant-made melanin nanoparticle precipitates in a plant extract without additional chemicals or adjustments by centrifugation, settling, or filtration.

9. A method of claim 1, wherein the plant-made melanin nanoparticle is precipitated by raising or lowering the pH or by enzymatic or chemical process.

10. A method of claim 1, wherein the melanin nanoparticle can be functionalized with a chemical, biochemical, DNA, RNA, aptamer, protein, enzyme, protectant, or other material that adds a function to the nanoparticle.

11. A use of claim 1, wherein the plant-made melanin nanoparticle can be utilized for industrial, biomedical, healthcare, agriculture, environmental, or other downstream use.

12. A use of claim 10, wherein the functionalized plant-made melanin nanoparticle can be used in healthcare, biotechnology, and biomedical such as a diagnostic or therapeutic, or other downstream use.
